(19) United States
(12) Patent Application Publication
(10) Pub. No.: US 2010/0035856 A1
(43) Pub. Date: Feb. 11, 2010

(54) Beta-LACTAM-CONTAINING FORMULATIONS WITH INCREASED STABILITY IN AQUEOUS SOLUTION

(75) Inventors: Dirk Mertin, Langenfeld (DE); Bernd Bigalke, Bergisch Gladbach (DE); Franz Pirro, Langenfeld (DE)

Correspondence Address: BAYER HEALTHCARE LLC, P.O.BOX 390, SHAWNEE MISSION, KS, 66201, US

(73) Assignee: BAYER ANIMAL HEALTH GMBH, LEVERKUSEN (DE)

(21) Appl. No.: 12/522,665

(22) PCT Filed: Jan. 10, 2008
(86) PCT No.: PCT/EP08/00125

§ 371 Date: Jul. 13, 2009

(30) Foreign Application Priority Data
Jan. 19, 2007 (DE) 102007002924.3

(51) Int. Cl. A61K 31/43; A61P 31/04

(52) U.S. Cl. 514/197; 514/192

(57) ABSTRACT

The invention relates to novel formulations for dissolution in water which contain a β-lactam antibiotic and urea and whose pH after dissolution of the formulation in water is in the range of from 4.5 to 8. The formulations are suitable in particular for the treatment of bacterial diseases in animals.

The invention relates to novel formulations for dissolution in water which contain a β-lactam antibiotic and urea and whose pH after dissolution of the formulation in water is in the range of from 4.5 to 8. The formulations are suitable in particular for the treatment of bacterial diseases in animals.

To treat bacterial infections in livestock, in particular poultry, pigs and calves, antibiotics are frequently dissolved in the drinking water to ensure simple and reliable administration. Among the active ingredients used here, the penicillin amoxicillin is of great importance. Such amoxicillin preparations are on the market, for example Vetrimoxin (Ceva), Suramox 50 (Virbac) and Amoxinsol 50 (Vetoquinol). With these preparations, the active ingredient is dissolved in the drinking water at a concentration of 100-300 ppm and this solution is fed into the livestock's drinking water supply. A more modern way of the administration via the drinking water is the preparation of an active-ingredient-containing concentrate which is continually fed into the animals' drinking water supply via a metering pump. However, by way of example, the preparation of such a concentrate with more than 0.3% m/V amoxicillin is not readily possible owing to the fact that the basic solubility of amoxicillin in water is poor.

However, amoxicillin, being a substance with an acidic carboxyl function and a basic amine function, can be dissolved by addition of equivalent amounts of acid or base. FIG. 1 shows the solubility of amoxicillin as a function of the pH.

By bringing the pH into an acidic (pH<3) or basic (pH>7) range, it is possible markedly to increase amoxicillin's solubility. However, amoxicillin is extremely sensitive to hydrolysis, as are other β-lactam antibiotics. A limited stabilization is possible by choosing an optimized pH range which, in the case of amoxicillin, is approximately between pH 5.0 and 7.0. Adjusting this pH range provides sufficient stability in drinking water. However, since amoxicillin's solubility is limited in such a case, an application as a concentrate (>0.3% m/V) has not been possible to date.

In an aqueous medium, β-lactams degrade hydrolytically, giving rise to oligomers. This is why the relative degradation rate is concentration-dependent (2nd-order reaction), which further reduces the stability of a concentrate.

It is known that the solubility of pharmaceutical active ingredients in water can be improved by addition of hydrotropic substances. Hydrotropism is understood as meaning the phenomenon that a sparingly soluble substance becomes water-soluble in the presence of a second component which itself is not a solvent. Substances which bring about such an improved solubility are referred to as hydrotropes or hydrotropics. They act as solubilizers with different mechanisms of action. Thus, for example, urea or N-methylacetamide increase the solubility by a structure-disintegrating effect, where the water structure in the environment of the hydrophobic group of a sparingly soluble substance is broken down.

The improvement of the solubility and of the rate of dissolution of β-lactam antibiotics as the result of the addition of urea has been described. Thus, Kwon et al. successfully improved the solubility of the cephalosporin 7-β-[(2)-2-(2-aminothiazol-4-yl)-2-methoxyiminoacetamido]-3-[(2,3-cyclopenteno-4-carbamoyl-1-pyridinium)methyl]-3-cephem-4-carboxylate sulphate (CKD-604) with the addition of sodium ascorbate, ascorbic acid and urea (Kwon S Y, Shin H J, Kim C K; Physicochemical characteristics of cephalosporin derivative, CKD-604: stabilization and solubilization in aqueous media; Yakche Hakhoechi (1999), 29(3), 205-210). Urea also improved the solubility and rate of dissolution of ampicillin in water (Jimenez F A, Sanchez-Morcillo J, Selles E; Effect of coadjuvants in the dissolution rate of oral ampicillin; Ciencia & Industria Farmaceutica (1979), 11(4), 175-80) and the bioavailability of ampicillin in rabbits (Jimenez F A, Sanchez-Morcillo J, Selles E; Effects of coadjuvants on the bioavailability of oral ampicillin: urea. Part II; Farmacia Clinica (1984), 1(8), 639-43, 645-7, 649-52). Furthermore, it was possible to improve ampicillin's rate of dissolution in artificial gastric or intestinal liquid by a solid dispersion in polyethylene glycol 4000 or 6000, urea or citric acid (Singhai S C, Mathur V B; Dissolution characteristics of ampicillin solid dispersions; Indian Drugs (1979), 16(10), 236-8). Machida et al. (JP 02124823, JP 02124822) described mixtures of cephalosporins with arginine, lysine, histidine, ornithine, citrulline, their hydrochlorides, hydroxyproline, sodium chloride, potassium chloride, urea, nicotinamide, sodium benzoate, sodium salicylate and/or taurine. Scharland (DE 2433424) studied the in-vitro release of urea-containing tablets with β-lactams, but without having quantified urea's effect on the release rate. The urea had been pretreated with methylene chloride here.

Urea, however, reduces the stability of β-lactam antibiotics. For example, amoxicillin/K clavulanate mixtures with urea will turn brown within a short period of time, even at room temperature. This incompatibility also becomes clear from microcalorimetric studies of such mixtures. FIG. 3 shows the evolution of heat of a 1:1 mixture of amoxicillin/K clavulanate 4:1 and urea in comparison with the two pure substances. The area under the curve is a measure for the extent of an exothermic decay reaction.

Surprisingly, however, it has now been found that the instability of such urea-containing β-lactam antibiotic formulations in aqueous solution is lower than expected. For example, while amoxicillin in an aqueous amoxiclav solution containing 40% urea is more unstable than in a corresponding solution without urea, the stability, with a loss of active ingredient of less than 10% within 24 hours, is considerably better than would have been expected on the basis of the microcalorimetric studies. Indeed, the stability of clavulanic acid was not adversely affected at all by the urea (FIG. 4).

The invention therefore relates to formulations for dissolution in water which contain a β-lactam antibiotic and urea, where the pH after dissolution of the formulation in water is in the range of from 4.5 to 8.

Despite urea's solubility-improving effect, the dissolution rate of the β-lactam may be too low, under practice conditions, depending on the concentration. In such a case, the β-lactam may initially be dissolved with a base, as a salt. Thereafter, the mixture is brought to the pH of the stability optimum using an acid or an acid donor. In this case, the urea prevents the precipitation of the β-lactam.

Moreover, it has been found that the preparations according to the invention are, after ready-to-drink solutions have been prepared, highly palatable and that, as a rule, an increase in the animals' weight gain can be found after administration.

The system according to the invention therefore consists of one or more β-lactam active ingredient(s) and urea. To increase the dissolution rate, it is possible to add a base and an acid, or acid donor. In accordance with a preferred embodiment, a substance is used which only gradually forms an acid; in this manner, it is possible simultaneously to dissolve all the components in water. Under the effect of the base, it is initially the β-lactam which dissolves; the liberation of acid, which starts simultaneously, then leads to the pH being brought to the desired stability optimum of the β-lactam.

The formulations are adjusted in such a way that the pH of the aqueous solution prepared therewith is in the range of from 4.5 to 8, preferably 5 to 7, especially preferably 5.5 to 6.5.

Examples of β-lactam active ingredient which can be used are: amoxicillin, ampicillin, azidocillin, azlocillin, aztreonam, benzylpenicillin, carbenicillin, cefaclor, cefadroxil, cefalexin, cefamandole, cefazolin, cefepim, cefixim, cefotaxime, cefotiam, cefoxitin, cefpodoxime, ceftazidime, ceftibuten, ceftriaxone, cefuroxime, cephaloridine, clavulanic acid, dicloxacillin, ertapenem, flucloxacillin, imipenem, latamoxef, loracarbef, meropenem, mezlocillin, oxacillin, phenoxymethylpenicillin, oxacillin, piperacillin, propicillin, sulbactam, sultamicillin, temocillin, ticarcillin. Preferred in this context are amoxicillin, ampicillin and clavulanic acid, with amoxicillin being especially preferred. The active ingredient combination of amoxicillin (in particular in the form of its trihydrate) and clavulanic acid (in particular in the form of its potassium salt), which is known per se and proven, is employed in accordance with an especially preferred embodiment. The mixing ratio amoxicillin/clavulanic acid, given as a mass ratio, is 10:1 to 1:1, preferably 8:1 to 2:1, especially preferably 4:1 to 2:1.

The β-lactam active ingredients can also be employed in the form of their pharmaceutically acceptable salts or esters or else as solvates, in particular hydrates, of the free acids, salts or esters.

The concentration of the β-lactam active ingredients in the formulations according to the invention is usually from 0.5 to 20% m/m, preferably from 1 to 10% m/m, especially preferably from 3 to 10% m/m.

The concentration of the β-lactam active ingredients in the aqueous solutions prepared from the formulations according to the invention is usually from 0.01 to 10% m/v, preferably from 0.1 to 10% m/V, especially preferably from 0.5 to 5% m/V (% m/V means g/100 ml solution).

Urea is usually employed in a concentration of 50-99% m/m, preferably 70-95% m/m and especially preferably 80-95% m/m in the formulations according to the invention. If an aqueous concentrate is prepared therefrom, the urea concentration is usually 1-90% m/V, preferably 10-60% m/V, and especially preferably 30-50% m/V, based on the aqueous solution.

The following substances may be used as the base: arginine, calcium carbonate, calcium hydrogen carbonate, potassium carbonate, potassium hydrogen carbonate, potassium hydrogen phosphate, potassium hydroxide, potassium phosphate, lysine, meglumine, morpholine, sodium acetate, sodium ascorbate, sodium benzoate, sodium borate, sodium butyrate, sodium caprate, sodium carbonate, sodium citrate, sodium formate, sodium gluconate, sodium glutamate, sodium hydrogen carbonate, sodium hydrogen phosphate, sodium hydroxide, sodium lactate, sodium malate, sodium maleate, sodium oxalate, sodium phosphate, sodium propionate, sodium pyruvate, sodium salicylate, sodium succinate, sodium tartrate, piperidine, triethylamine, trometamol. Preferred in this context are arginine, potassium carbonate, lysine, meglumine, sodium carbonate, sodium hydroxide, sodium phosphate and trometamol.

Examples of acids which may be used are: adipic acid, formic acid, malic acid, ascorbic acid, aspartic acid, benzoic acid, succinic acid, boric acid, pyruvic acid, butyric acid, caproic acid, citric acid, acetic acid, galacturonic acid, gluconic acid, glucuronic acid, glutamic acid, gulonic acid, maleic acid, malonic acid, mannuronic acid, lactic acid, oxalic acid, phosphoric acid, phthalic acid, propionic acid, nitric acid, hydrochloric acid, sulphuric acid, sulphurous acid, sulphonic acid, tartaric acid. Acid-liberating derivatives which may be employed are, for example, esters, lactones, anhydrides, for example galacturonolactone, gluconolactone, glucuronolactone, gulonolactone, lactide, maleic anhydride, mannuronolactone, phthalic anhydride.

Substances which are preferably employed for the reasons already mentioned are acid derivatives which liberate the acid in a delayed manner; these are, in particular, lactones; examples which can be used are the following: galacturonolactone, gluconolactone, glucuronolactone, gulonolactone, lactide or mannuronolactone. Especially preferred in this context is glucono-δ-lactone (D-gluconic acid, 5-lactone), which, in water, slowly hydrolyses to give gluconic acid.

Since the urea may adversely affect the stability of the beta-lactam, the formulations according to the invention can, in accordance with a preferred embodiment, be divided into two components: one component contains the β-lactam and, if appropriate, the base or the acid-forming substance. The other component contains the urea and the acid, or the acid-forming substance (if the first component contains the base) or, if appropriate, the base (if the first component contains the acid-forming substance). However, it is also possible to mix the β-lactam with acid or acid-forming substance and base and to separate the urea therefrom. However, preferred two-component systems are those embodiments in which acid or acid-forming substance and base are separate. To prepare the aqueous solution, the two components are dissolved in water.

If an acid is used to adjust the pH, it is advantageous in the two-component system first to dissolve the β-lactam component and then to add the urea/acid component. If the acid is only formed from an acid-liberating substance in a time-delayed manner, the two components may also simply be dissolved in water at the same time.

If the β-lactam is sufficiently stable, or if this can be achieved by other measures known per se, formulations with acid-liberating substances are also highly suitable for single-component systems

Before the dissolution in water, the formulations according to the invention, or the components, are preferably present in solid form, for example as powders, granules, pellets or tablets. The formulations, or the components, are usually prepared in a known manner by mixing the constituents and, if appropriate, further processing such as grinding, granulating, tableting or the like. To prepare a ready-to-use composition, the formulations are usually dissolved in water in such a way that the β-lactam concentration is in the range of from 0.01 to 0.1% m/V. Frequently, however, a concentrate will be prepared by dissolution in water, and this concentrate can then be metered into the drinking water or the food. The β-lactam concentration in a concentrate is usually in the range of from 0.5 to 10% m/V.

The antibacterial activity of the β-lactams is known per se. The formulations according to the invention, or the aqueous solutions obtainable therefrom, can be employed accordingly.

The formulations according to the invention and the aqueous solutions obtainable therefrom are generally suitable for application in humans and animals. They are preferably employed in animal keeping and animal breeding in livestock, breeding animals, zoo animals, laboratory animals, experimental animals and pets.

The livestock and breeding animals include mammals, such as, for example, cattle, horses, sheep, pigs, goats, camels, water buffalos, donkeys, rabbits, fallow deer, reindeer, fur-bearers such as, for example, mink, chinchillas, racoons, and birds such as, for example, chickens, geese, turkeys, ducks, pigeons and bird species which are kept on domestic premises and in zoos.

Laboratory and experimental animals include mice, rats, guineapigs, golden hamsters, dogs and cats.

Pets include rabbits, hamsters, guineapigs, mice, horses, reptiles, suitable bird species, dogs and cats.

Fish may furthermore be mentioned; these are commercial fish, farmed fish, aquarium fish and ornamental fish of all ages which live in fresh water and in salt water.

Preferred is the use in poultry, for example geese, ducks, pigeons and in particular turkeys and chickens, and in pigs and calves.

The application can be both prophylactic and therapeutic.

The formulations described herein are usually administered after dissolution in water and, if appropriate, further dilution, preferably via the oral route.

### EXAMPLES

### Example 1

30 g of amoxicillin trihydrate/potassium clavulanate 4:1 (mixture of amoxicillin trihydrate and K-clavulanate according to a mass ratio of 4 parts of anhydrous amoxicillin and 1 part of clavulanic acid) and 13 g of gluconolactone on the one hand and 4.0 g of sodium carbonate and 400 g of urea on the other hand are mixed and jointly dissolved in water to give an end volume of 1000 ml. This gives a concentrate with 2% m/V amoxicillin (anhydrous) and 0.5% m/V clavulanic acid.

### Example 2

30 g of amoxicillin trihydrate/K-clavulanate 4:1 and 6.5 g of gluconolactone on the one hand and 1.3 g of sodium hydroxide and 400 g of urea on the other hand are mixed and jointly dissolved in water to give an end volume of 1000 ml. This gives a concentrate with 2% m/V amoxicillin (anhydrous) and 0.5% m/V clavulanic acid.

### Example 3

30 g of amoxicillin trihydrate/K-clavulanate 4:1 are mixed with 6.0 g of tert.-sodium phosphate. 400 g of urea and 12.5 g of gluconolactone are mixed in a separate container. The two mixtures are jointly dissolved in water to give an end volume of 1000 ml. This gives a concentrate with 2% m/V amoxicillin (anhydrous) and 0.5% m/V clavulanic acid.

### Example 4

30 g of amoxicillin trihydrate/K-clavulanate 4:1 are mixed with 7.0 g of gluconolactone. 6.4 g of arginine and 400 g of urea are mixed in a separate container. The two mixtures are jointly dissolved in water to give an end volume of 1000 ml. This gives a concentrate with 2% m/V amoxicillin (anhydrous) and 0.5% m/V clavulanic acid.

### Example 5

30 g of amoxicillin trihydrate/K-clavulanate 4:1 and 5.4 g of lysine on the one hand and 400 g of urea and 7.0 g of gluconolactone on the other hand are mixed and jointly dissolved in water to give an end volume of 1000 ml. This gives a concentrate with 2% m/V amoxicillin (anhydrous) and 0.5% m/V clavulanic acid.

### Example 6

30 g of amoxicillin trihydrate/K-clavulanate 4:1 and 7.2 g of meglumine on the one hand and 400 g of urea and 7.0 g of gluconolactone on the other hand are jointly dissolved in water to give an end volume of 1000 ml. This gives a concentrate with 2% m/V amoxicillin (anhydrous) and 0.5% m/V clavulanic acid.

### Example 7

30 g of amoxicillin trihydrate/K-clavulanate 4:1 and 4.5 g of trometamol on the one hand and 400 g of urea and 7.0 g of gluconolactone on the other hand are jointly dissolved in water to give an end volume of 1000 ml. This gives a concentrate with 2% m/V amoxicillin (anhydrous) and 0.5% m/V clavulanic acid.

### Example 8

30 g of amoxicillin trihydrate/K-clavulanate 4:1, 4.0 g of sodium carbonate and 13 g of gluconolactone are mixed. This mixture together with 400 g of urea is dissolved in water to give an end volume of 1000 ml. This gives a concentrate with 2% m/V amoxicillin (anhydrous) and 0.5% m/V clavulanic acid.

### Example 9

400 g of urea, 6.4 g of arginine and 7.0 g of gluconolactone are mixed. This mixture together with 30 g of amoxicillin trihydrate/K-clavulanate 4:1 is dissolved in water to give an end volume of 1000 ml. This gives a concentrate with 2% m/V amoxicillin (anhydrous) and 0.5% m/V clavulanic acid.

### Example 10

14.7 g of amoxicillin trihydrate/K-clavulanate 4:1, 3.5 g of gluconolactone and 3.2 g of arginine are mixed and together with 200 g of urea dissolved in 50 litres of water. This gives a ready-to-drink solution with 200 ppm amoxicillin (anhydrous), 50 ppm clavulanic acid and 4000 ppm urea.

### Example 11

29.4 g of amoxicillin trihydrate/K-clavulanate 4:1, 7.0 g of gluconolactone and 6.4 g of arginine are mixed and together with 400 g of urea dissolved in 50 litres of water. This gives a ready-to-drink solution with 400 ppm amoxicillin (anhydrous), 100 ppm clavulanic acid and 8000 ppm urea.

### Example 12

30 g of amoxicillin trihydrate/K-clavulanate 4:1 and 13.5 g of arginine are mixed and dissolved in 900 ml of water. 400 g of urea and 5.6 g of tartaric acid are mixed in a separate container and added to the solution. The solution is made up to 1000 ml with water.

FIG. 5 and FIG. 6 show the stability of the active ingredients of Examples 2 and 4-7.

### BIOLOGICAL EXAMPLE

### Example A

Acceptance of drinking water solutions containing amoxicillin trihydrate/clavulanic acid in turkeys

Three groups of in each case 80 turkeys were given the following drinking water solutions over a total of 8 weeks:

1. Drinking water without addition

2. 200/50 ppm amoxicillin/clavulanic acid+4000 ppm urea (Example 10)

3. 400/100 ppm amoxicillin/clavulanic acid+8000 ppm urea (Example 11)

4. 4000 ppm urea

5. 8000 ppm urea

The daily drinking water consumption per animal group was determined over in each case four treatment phases of two weeks each. The drinking water consumption is a measure for the palatability of the drinking water preparation in question. The turkeys' consumption of the drinking water solutions containing amoxicillin/clavulanic acid is shown in FIG. 7.

Examples 10 and 11 according to the invention show a higher drinking water consumption and therefore better palatability than urea solutions without active ingredient, or unmedicated drinking water.

FIG. 8 shows that the turkeys' weight gain while administering Examples 10 and 11 according to the invention is also increased.

### FIGURES

FIG. 1: Solubility of amoxicillin as a function of the pH

FIG. 2: Stability of amoxicillin as a function of the pH

FIG. 3: Microcalorimetric study of amoxicillin/K-clavulanate (4:1), urea and an amoxicillin/K-clavulanate (4:1)-urea mixture (mixing ratio 1:1)

FIG. 4: Effect of urea on the stability of an 0.3% solution of amoxicillin/K-clavulanate 4:1 in water, pH 6.5

FIG. 5: Stability of amoxicillin in aqueous solution at room temperature in accordance with Examples 2 and 4-7

FIG. 6: Stability of clavulanic acid in aqueous solution at room temperature in accordance with Examples 2 and 4-7

FIG. 7: Consumption by turkeys (n=240) of drinking water solutions containing amoxicillin/clavulanic acid

FIG. 8: Turkey weight after 57 days' treatment with drinking water solutions containing amoxicillin/clavulanic acid, and comparative solutions (n=90)

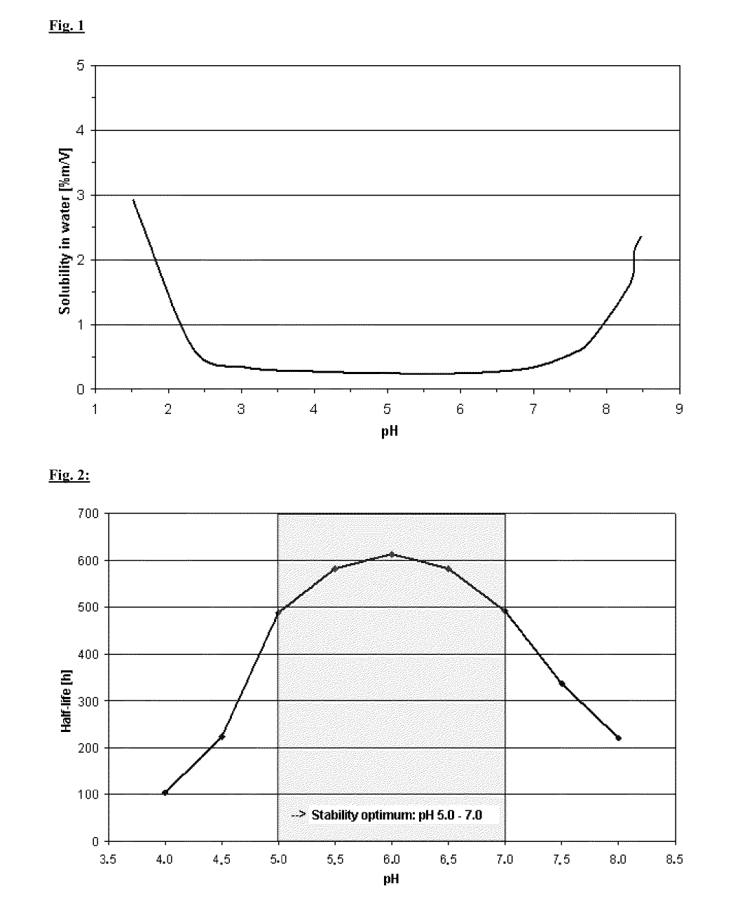

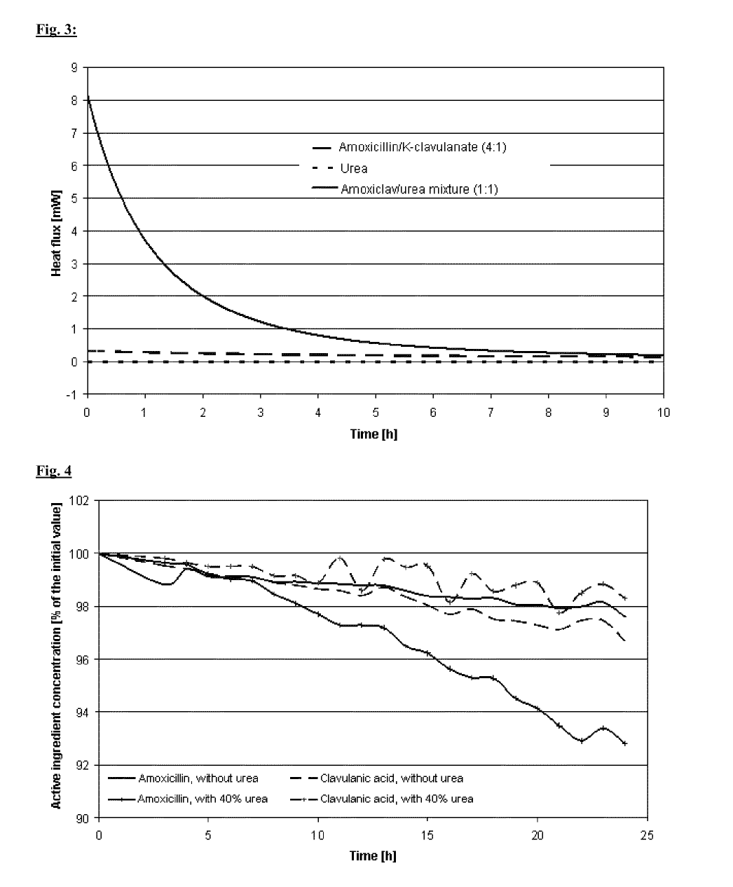

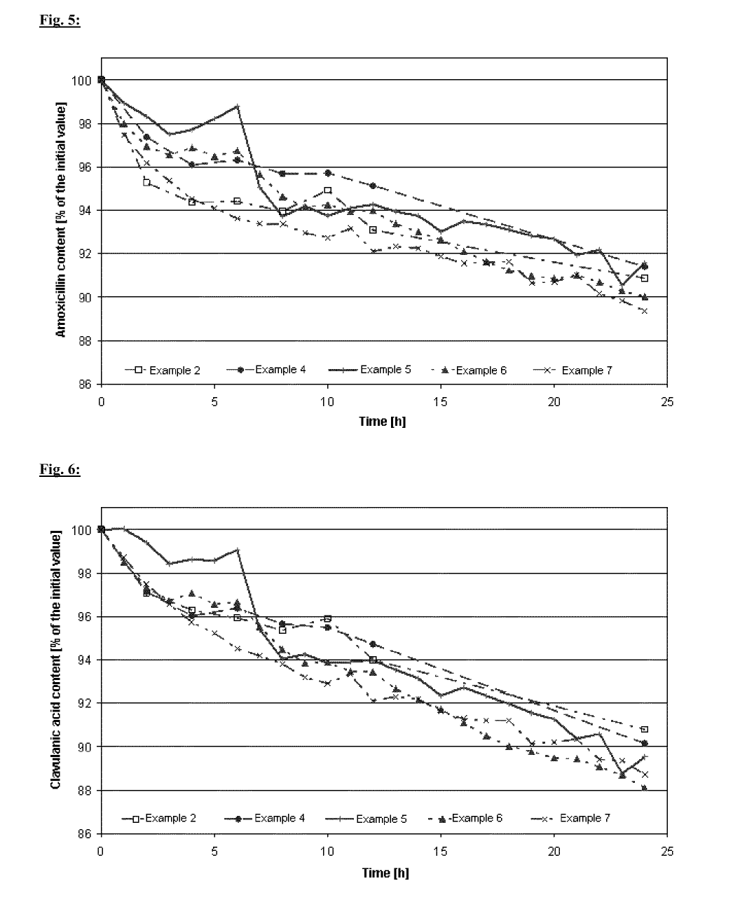

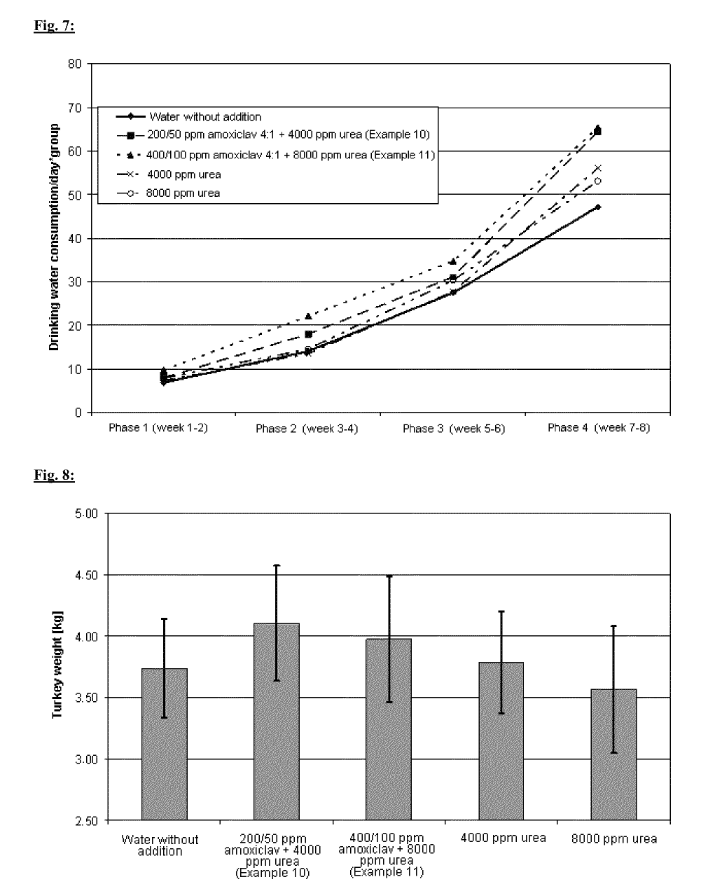

1. A formulation for dissolution in water which contains a β lactam antibiotic and urea, where the pH after dissolution of the formulation in water is in the range of from 4.5 to 8.

2. The formulation of claim 1, wherein the β lactam antibiotic can be dissolved in water at a concentration of more than 0.3% m/V.

3. The formulation of claim 1, wherein the formulation further comprises a base.

4. The formulation of claim 1, wherein the formulation further comprises a base and an acid or an acid derivative.

5. The formulation of claim 3, wherein the base is selected from the group consisting of arginine, potassium carbonate, lysine, meglumine, sodium carbonate, sodium hydroxide, sodium phosphate and trometamol.

6. The formulation of claim 5, wherein the base is arginine.

7. The formulation of claim 4, wherein the acid derivative is a lactone.

8. The formulation of claim 7, wherein the lactone is glucono-δ-lactone.

9. The formulation of claim 1, wherein the β lactam antibiotic is amoxicillin.

10. The formulation of claim 9, wherein the amoxicillin is amoxicillin trihydrate and the formulation further comprises clavulanic acid or a clavulanic acid salt.

11. (canceled)

12. (canceled)

13. (canceled)

14. (canceled)

15. A process for the preparation of a ready-to-use aqueous composition containing a β lactam antibiotic and urea, the process comprising:
a. mixing the β lactam antibiotic with a base, acid, or acid-forming substance to form a β lactam formulation;
b. mixing the urea with an acid, acid-forming substance, or base to form a urea formulation;
c. dissolving the β lactam formulation and the urea formulation jointly in water to form a ready-to-use composition, and
wherein if the base is mixed with the β lactam antibiotic, the acid or acid-forming substance is mixed with the urea, and if the acid-forming substance is mixed with the β lactam antibiotic, the base is mixed with the urea, and wherein the composition has a β lactam antibiotic concentration of from 0.01 to 0.1% m/V.

16. A pharmaceutical composition containing a β lactam antibiotic and urea comprising:
a. a β lactam antibiotic;
b. a urea;
c. and water; wherein the β lactam antibiotic concentration is from 0.01 to 0.1% m/V.

17. The pharmaceutical composition of claim 16, wherein the β lactam antibiotic is amoxicillin.

18. (canceled)

19. (canceled)

20. A method for treating a bacterial infection in an animal, the method comprising administering to an animal in need thereof an effective amount of the composition of claim 16.

21. The pharmaceutical composition of claim 16, wherein the composition is a concentrate and wherein the β lactam antibiotic concentration is from 0.5 to 10% m/V.

22. The composition of claim 21, wherein the urea concentration is from 30 to 50% m/V.

23. The formulation of claim 1, wherein the β lactam antibiotic concentration is from 3 to 10% m/m.

24. The formulation of claim 1, wherein the urea concentration is from 80 to 95% m/m.
